(19) United States
(12) Patent Application Publication
(10) Pub. No.: US 2026/0263770 A1
(43) Pub. Date: Sep. 10, 2026

(54) Enforced Tissue Residency of Payload Molecules By Acoustic Disruption

(71) Applicant: Insightec Ltd., Tirat Carmel (IL)

(72) Inventors: Eyal ZADICARIO, Tel Aviv-Yafo (IL); Yoav LEVY, Hinanit (IL); Michael PLAKSIN, Tirat Carmel (IL); Tiran BERCOVICI, Tirat Carmel (IL); Gabriella TOLTSIS, Shimshit (IL)

(21) Appl. No.: 18/995,563

(22) PCT Filed: Aug. 31, 2023
(86) PCT No.: PCT/IB2023/058628

§ 371 Date: Jan. 16, 2025

Related U.S. Application Data
(60) Provisional application No. 63/402,493, filed on Aug. 31, 2022.
(60) Provisional application No. 63/453,924, filed on Mar. 22, 2023.

(51) Int. Cl. A61M 37/00

(52) U.S. Cl. CPC A61M 37/0092; A61M 2210/0693

(57) ABSTRACT

A system (100) for controlling drug delivery through a tissue barrier includes an ultrasound transducer (104) and a controller (108). The ultrasound transducer is for sonicating a target volume to cause disruption of the tissue barrier and thereby increase a permeability thereof. The controller is configured to receive a specification of a target molecule, where the specification includes a pharmacokinetic parameter of the target molecule or a molecular size of the target molecule. The controller is also configured to operate the ultrasound transducer in accordance with the specification to permit passage of the target molecule through the tissue barrier in a manner that traps a target fraction of the target molecule behind the tissue barrier for a predetermined time period.

### REFERENCE TO RELATED APPLICATIONS

This application claims priority to U.S. Provisional Patent Application Nos. 63/402,493 (filed Aug. 31, 2022) and 63/453,924 (filed Mar. 22, 2023), each of which is hereby incorporated by reference in its entirety.

### TECHNICAL FIELD

The present disclosure relates, generally, to systems and methods for ultrasound focusing and, more particularly, to controlling drug delivery through tissue barriers.

### BACKGROUND

Focused ultrasound has been widely explored as a method for permeabilizing tissue barriers, such as the blood-brain barrier (BBB). Permeabilization allows drugs to move through these barriers more freely, thus allowing for medication of tissue that would otherwise be difficult or impossible to reach without invasive surgery. Focused ultrasound (i.e., acoustic waves with frequencies greater than approximately 20 kilohertz) can be used in various therapies, for example, targeted drug delivery, control of the BBB, lysing of clots, neuromodulation, ablation of tumors, and other clinical procedures.

During a focused ultrasound procedure, a series of sonications is applied to affect a target tissue. The sonications may cause death of the target tissue (such as a tumor) without damaging surrounding tissue, disrupt the BBB for targeted delivery of therapeutic agents, be used in neuromodulation treatments, and so forth. To achieve these outcomes, ultrasonic energy emitted from the transducer must be accurately and reliably shaped and focused onto the desired target location. There remains a challenge, however, to optimizing the dosage and stability of drugs once they reach their target in the brain in order to maximize treatment efficiency and minimize the number of necessary treatment procedures.

The BBB and other tissue barriers can impede the transmission of therapeutic agents and biomarkers used in medical treatment. For example, the BBB impedes the transmission of molecules with a molecular weight greater than 400 daltons from the blood to the brain parenchyma. This diminishes the effectiveness of a vast majority of neural therapeutic agents and biomarkers.

Fortunately, ultrasound can overcome the impedances posed by tissue barriers. It enables the transfer of different-sized molecules, for example, through the BBB, from one nanometer-sized molecules (e.g., Omiscan, Gadavist, and Dotarem), to tens of nanometers-sized molecules (e.g., fluorescein-tagged dextrans, gold nanoparticles, adeno-associated viral vectors, and liposomal doxorubicin), and even neural stem cells several microns in size.

Despite significant developments in the ultrasound field, current technologies generally require specific acoustic parameters that must be met for the effective transfer of molecules through the BBB. Further, current technologies struggle to predict the quantity of the therapeutic agent that will effectively reach the targeted area during and after the BBB-opening treatment.

### SUMMARY

The present disclosure provides systems and methods that address the aforenoted problems. Some of the systems and methods described herein, for example, utilize physical parameters to measure and predict the effectiveness of BBB-opening during treatment. This is discussed in more detail below.

In one embodiment, a tissue barrier (e.g., the BBB) is opened transiently to allow for large molecules (e.g., larger than 1 nanometer in diameter) to pass through the tissue barrier into a tissue region (e.g., the brain parenchyma). For example, this may allow for the treatment of malignancies (e.g., glioblastoma) or neurological disorders (e.g., Alzheimer's disease). These molecules may remain trapped in the tissue after the BBB closes and thus can continue to exert their therapeutic effects in the treated area. The systems and methods described herein are suited for use with large molecules having a long half-life or large molecular vehicles (e.g., viral vectors, liposomes, micelles) engineered for slow drug release.

In another embodiment, a system controls drug delivery through a tissue barrier. The system comprises an ultrasound transducer for sonicating a target volume—also referred to as a target tissue or a target tissue region—to cause disruption of the target tissue volume and thereby increase its permeability. The system may also include a controller configured to (i) receive a specification of a target molecule, with the specification including at least one pharmacokinetic parameter or a molecular size of the target molecule, and (ii) operate the ultrasound transducer in accordance with the specification to permit passage of the target molecule through the tissue barrier in a manner that traps a target fraction of the target molecule behind the barrier for a predetermined time period.

The target molecule may be injected before or after operation of the ultrasound transducer has been initiated. Additionally, the transducer may be operated in accordance with a sonication protocol to temporarily permeabilize the tissue barrier to the molecule, whereby the tissue barrier becomes impermeable to the molecule prior to its clearance into the bloodstream from behind the tissue barrier. Moreover, the transducer may be operated in accordance with a sonication protocol to partially permeabilize the tissue barrier to the molecule, whereby the target fraction remains behind the tissue barrier for the predetermined time period. Furthermore, the molecule may undergo a one-way transition in size, conformation or chemical properties following passage through the tissue barrier so that it is retained behind the barrier.

In general, ultrasonic energy may be focused to a zone having a cross-section of only a few millimeters due to relatively short wavelengths (e.g., 1.5 millimeters at 1 megahertz). Moreover, because acoustic energy generally penetrates well through soft tissues, intervening anatomy often does not pose an obstacle to defining a desired focal zone. Thus, ultrasonic energy may be focused at a small target in order to ablate diseased tissue without significantly damaging surrounding healthy tissue, or to selectively open the BBB in the focus region using blood stream acoustic contrast agents.

An ultrasound focusing system generally utilizes an acoustic transducer surface or an array of transducer surfaces to generate an ultrasound beam. The transducer may be geometrically shaped and positioned to focus the ultrasonic energy at a “focal zone” corresponding to a target tissue mass within the patient. During wave propagation through the tissue, a portion of the ultrasound energy is absorbed, leading to increased temperature and, eventually, to cell death—preferably at the target tissue mass in the focal zone, or BBB opening using blood stream acoustic contrast agents.

The individual surfaces, or “elements,” of the transducer array can be individually controllable. For example, the phases and/or amplitudes of the individual elements can be set independently of one another. This may be accomplished, for instance, using a “beamformer” with suitable delay or phase shift in the case of continuous waves and amplifier circuitry for the elements, allowing the beam to be steered in a desired direction and focused at a desired distance, and the focal zone properties to be shaped as needed. Thus, the focal zone can be rapidly displaced and/or reshaped by independently adjusting the amplitudes and/or phases of the electrical signal input into the transducer elements.

It is noted that the opening of the tissue barrier may be tailored so as to maximize the final concentration in tissue of the target molecule based on, for example, the drug-clearance rate, the recovery rate of the tissue barrier (e.g., how quickly it reverts to an impermeable state following ultrasound exposure), or other kinetic parameters.

### BRIEF DESCRIPTION OF THE DRAWINGS

In the drawings, like reference characters generally refer to the same parts throughout the different views. Also, the drawings are not necessarily to scale, with an emphasis instead generally being placed upon illustrating the principles of this disclosure. In the following description, various embodiments of the present disclosure are described with reference to the following drawings, in which:

FIG. 1 depicts an exemplary ultrasound system for generating and delivering a focused acoustic energy beam to a target region within a patient's body, in accordance with various embodiments of the present disclosure.

FIG. 2 depicts an exemplary imager, namely, an MRI apparatus 200, in accordance with various embodiments of the present disclosure.

FIGS. 3A-3C are exemplary line plots that depict a relationship between time and a trapped concentration of a molecule in tissue, in accordance with various embodiments of the present disclosure.

FIGS. 4A-4C are exemplary coronal gradient echo sequences that depict rat brains following a BBB-opening treatment, in accordance with various embodiments of the present disclosure.

FIGS. 5A-5B are exemplary image sets that depict rat brains with different-sized liposomes introduced to respective brain parenchyma, in accordance with various embodiments of the present disclosure.

FIG. 6 is another exemplary set of images that depict pig brains with liposomes introduced to respective brain parenchyma, in accordance with various embodiments of the present disclosure.

FIG. 7 is an exemplary block diagram that depicts a process for controlling drug delivery through a tissue barrier, in accordance with various embodiments of the present disclosure.

### DETAILED DESCRIPTION

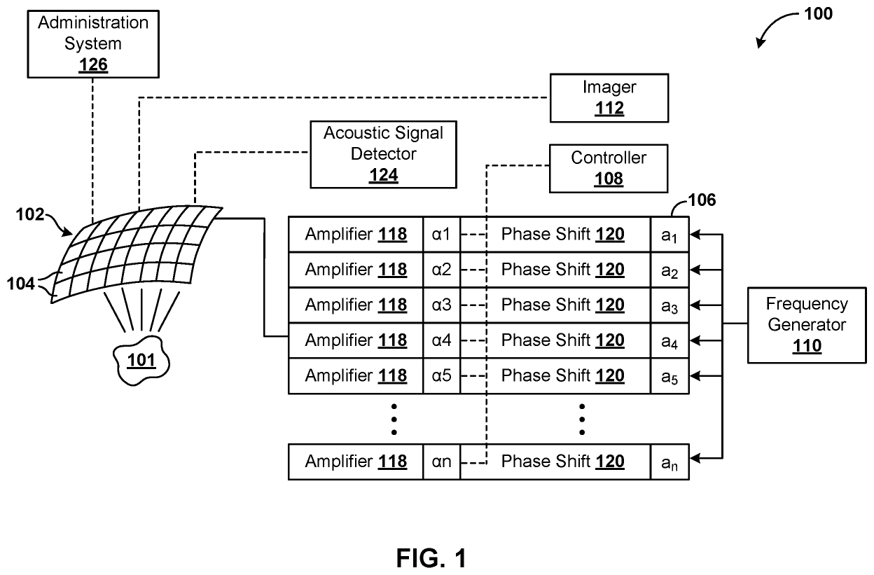

FIG. 1 depicts an exemplary ultrasound system 100 for generating and delivering a focused acoustic energy beam to a target region 101 within a patient's body, in accordance with various embodiments of the present disclosure. The illustrated system 100 includes a phased array 102 of transducer elements 104, a beamformer 106 driving the phased array 102, a controller 108 in communication with the beamformer 106, and a frequency generator 110 providing an input electronic signal to the beamformer 106.

The array 102 may have a curved (e.g., spherical or parabolic) or other contoured shape suitable for placement on the surface of the patient's body, or may include one or more planar or otherwise shaped sections. Its dimensions may vary between millimeters and tens of centimeters. The transducer elements 104 of the array 102 may be piezoelectric ceramic elements, and may be mounted in silicone rubber or any other material suitable for damping the mechanical coupling between the elements 104. Piezo-composite materials, or generally any materials capable of converting electrical energy to acoustic energy, may also be used. To assure maximum power transfer to the transducer elements 104, the elements 104 may be configured for electrical resonance at 50Ω, matching input connector impedance.

The transducer array 102 is coupled to the beamformer 106, which drives the individual transducer elements 104 so that they collectively produce a focused ultrasonic beam or field. For n transducer elements, the beamformer 106 may contain n driver circuits, each including or consisting of an amplifier 118 and a phase delay circuit 120; each drive circuit drives one of the transducer elements 104. The beamformer 106 receives a radio frequency (RF) input signal, typically in the range from 0.1 MHz to 10 MHz, from the frequency generator 110, which may, for example, be a Model DS345 generator available from Stanford Research Systems. The input signal may be split into n channels for the n amplifiers 118 and delay circuits 120 of the beamformer 106. In some embodiments, the frequency generator 110 is integrated with the beamformer 106. The radio frequency generator 110 and the beamformer 106 are configured to drive the individual transducer elements 104 of the transducer array 102 at the same frequency, but at different phases and/or different amplitudes.

The amplification or attenuation factors al-an and the phase shifts al-an imposed by the beamformer 106 serve to transmit and focus ultrasonic energy through the intervening tissue located between the transducer elements 104 and the target region onto the target region 101, and account for wave distortions induced in the intervening tissue. The amplification factors and phase shifts are computed using the controller 108, which may provide the computational functions through software, hardware, firmware, hardwiring, or any combination thereof.

In various embodiments, the controller 108 utilizes a general-purpose or special-purpose digital data processor programmed with software in a conventional manner, and without undue experimentation, to determine the frequency, phase shifts and/or amplification factors necessary to obtain a desired focus or any other desired spatial field patterns at the target region 101. In certain embodiments, the computation is based on detailed information about the characteristics (e.g., the type, size, location, property, structure, thickness, density, structure, etc.) of the intervening tissue located between the transducer element 104 and the target and their effects on propagation of acoustic energy. Such information may be obtained from an imager 112. The imager 112 may be, for example, an MRI device, a computer tomography (CT) device, a positron emission tomography (PET) device, a single-photon emission computed tomography (SPECT) device, or an ultrasonography device. Image acquisition may be three-dimensional (3D) or, alternatively, the imager 112 may provide a set of two-dimensional (2D) images suitable for reconstructing a three-dimensional image of the target region 101 and/or other regions (e.g., the region surrounding the target 101 or another target region).

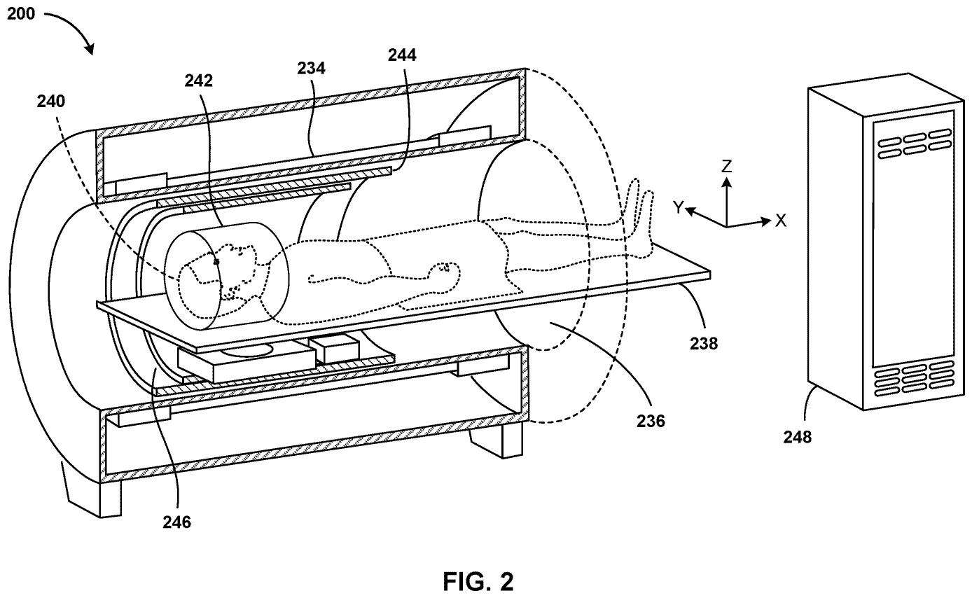

Image-manipulation functionality may be implemented in the imager 112, in the controller 108, or in a separate device. In addition, the ultrasound system 100 and/or imager 112 may be utilized to detect signals from an acoustic reflector (e.g., microbubbles 202, see FIG. 2) located substantially close to the target region 101 as further described below. As such, the system 100 may include a device configured to administer an acoustic agent (also referred to as an acoustic reflector) in the form of microbubbles, nanobubbles, or phase-shift droplets. Additionally or alternatively, the system 100 may include an acoustic-signal detection device (such as a hydrophone or suitable alternative) 124 that detects transmitted or reflected ultrasound from the acoustic reflector, and which may provide the signals it receives to the controller 108 for further processing. In addition, the ultrasound system 100 may include an administration system 126 for parenterally introducing the acoustic reflector into the patient's body. The imager 112, the acoustic-signal detection device 124, and/or the administration system 126 may be operated using the same controller 108 that facilitates the transducer operation; alternatively, they may be separately controlled by one or more separate controllers intercommunicating with one another.

FIG. 2 depicts an exemplary imager (e.g., imager 112 of FIG. 1), namely, an MRI apparatus 200, in accordance with various embodiments of the present disclosure. The apparatus 200 may include a cylindrical electromagnet 234, which generates the requisite static magnetic field within a bore 236 of the electromagnet 234. During medical procedures, a patient is placed inside the bore 236 on a movable support table 238. A region of interest 240 within the patient (e.g., the patient's head) may be positioned within an imaging region 242 wherein the electromagnet 234 generates a substantially homogeneous field. A set of cylindrical magnetic field gradient coils 244 may also be provided within the bore 236 and surrounding the patient. The gradient coils 244 generate magnetic field gradients of predetermined magnitudes, at predetermined times, and in three mutually orthogonal directions. With the field gradients, different spatial locations can be associated with different precession frequencies, thereby giving a magnetic-resonance (MR) image its spatial resolution. An RF transmitter coil 246 surrounding the imaging region 242 emits RF pulses into the imaging region 242 to cause the patient's tissues to emit MR response signals. Raw MR response signals are sensed by the RF coil 246 and passed to an MR controller 248 that then computes an MR image, which may be displayed to the user. Alternatively, separate MR transmitter and receiver coils may be used. Images acquired using the MRI apparatus 200 may provide radiologists and physicians with a visual contrast between different tissues and detailed internal views of a patient's anatomy that cannot be visualized with conventional x-ray technology.

The MRI controller 248 may control the pulse sequence, i.e., the relative timing and strengths of the magnetic field gradients and the RF excitation pulses and response detection periods. The MR response signals are amplified, conditioned, and digitized into raw data using a conventional image-processing system, and further transformed into arrays of image data by methods known to those of ordinary skill in the art. Based on the image data, the target region (e.g., a tumor or a target BBB) can be identified.

To perform targeted drug delivery or tumor ablation, it is necessary to determine the location of the target region 101 with high precision. Accordingly, in various embodiments, the MRI apparatus 200 is first activated to acquire images of the target region 101 and/or non-target region (e.g., the healthy tissue surrounding the target region, the intervening tissue located between the transducer array 102 and the target region 101 and/or any regions located near the target) and, based thereon, determine anatomical characteristics (e.g., the tissue type, location, size, thickness, density, structure, shape, vascularization) associated therewith. For example, a tissue volume may be represented as a 3D set of voxels based on a 3D image or a series of 2D image slices and may include the target region 101 and/or non-target region.

To create a high-quality focus at the target region 101, it may be necessary to calibrate the transducer elements 104 and take into account transducer geometric imperfections resulting from, for example, movement, shifts and/or deformation of the transducer elements 104 from their expected locations. In addition, because the ultrasound waves may be scattered, absorbed, reflected and/or refracted when traveling through inhomogeneous intervening tissues located between the transducer elements 104 and the target region 101, accounting for these wave distortions may also be necessary in order to improve the focusing properties at the target region 101.

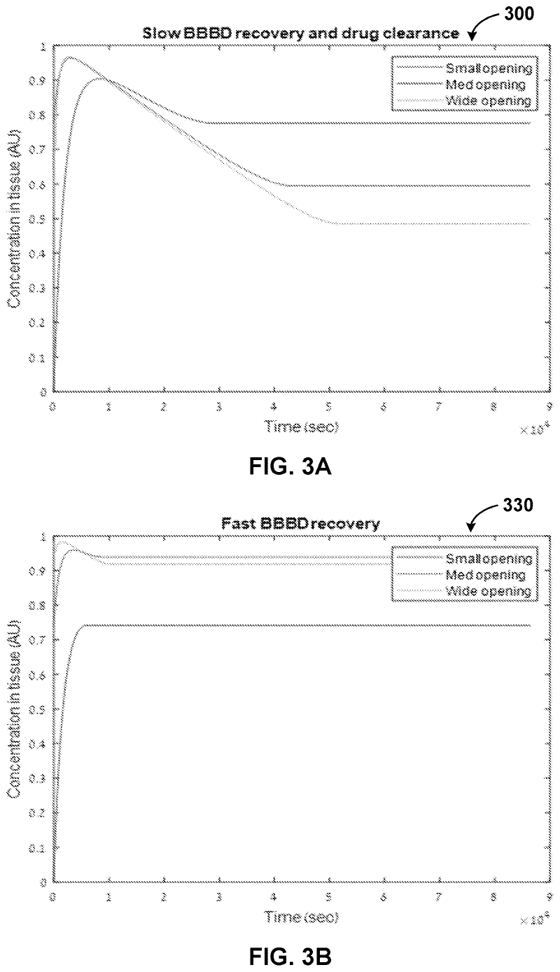

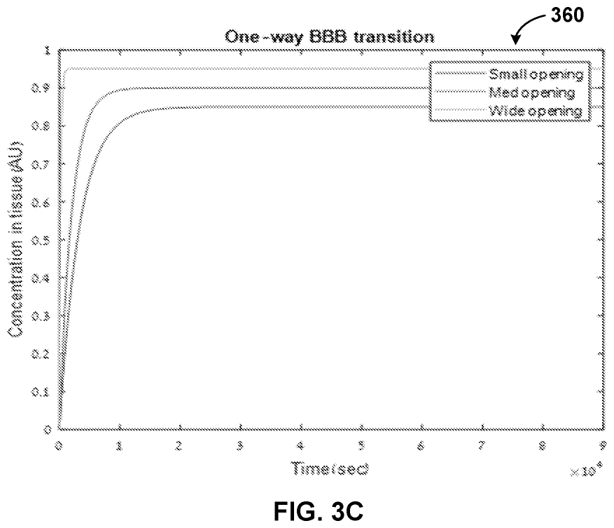

FIGS. 3A-3C are exemplary line plots 300, 330, and 360 that each depict an amount of a trapped molecule in tissue over time, in accordance with various embodiments of the present disclosure. In each figure, plotted lines correspond to small, medium, and wide openings, thus illustrating the effect of various opening sizes on the amount of the trapped molecule over time. In particular, FIG. 3A relates to a tissue barrier with a slow recovery rate. FIG. 3B relates to a tissue barrier with a fast recovery rate. And FIG. 3C relates to a tissue barrier allowing one-way transition of the molecule, for example, due to size or chemical changes that can occur after delivery of the molecule to the brain parenchyma.

The line plots 300, 330, and 360 of FIGS. 3A-3C, respectively, illustrate two somewhat extreme situations: In the first line plot 300, the concentration falls considerably due to how long the tissue barrier remains permeable. This permeability enables circulation to remove a considerable fraction of the molecule that has crossed the tissue barrier. By contrast, as illustrated in the second and third line plots 330 and 360, if the tissue barrier closes more quickly or permits one-way transition, a much larger fraction of the molecule remains trapped.

Hence, measures to improve trapping efficiency include changing the size of the target molecule to limit its clearance rate (e.g., tailoring the size by attaching several drug molecules one to another or alternatively attaching the drug molecules to a complexing agent), changing the surface chemistry of the molecules to form aggregates after parenchymal delivery, altering the sonication protocol to change the degree of barrier opening, and administering a drug that changes the closure rate of the barrier. Improvements in trapping efficiency result in the creation of a drug reservoir in the brain after tissue barrier opening and closing.

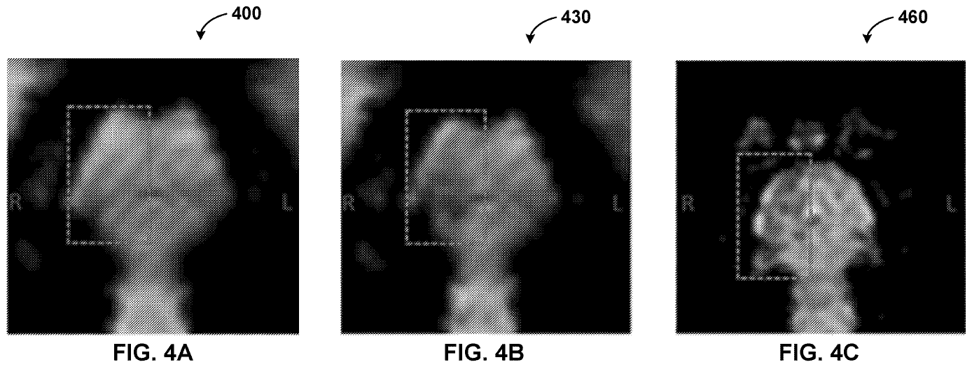

FIGS. 4A-4C are exemplary coronal gradient echo sequences 400, 430, and 460 that depict rat brains following a BBB-opening treatment, in accordance with various embodiments of the present disclosure. The BBB-opening treatment can create a drug reservoir for iron oxide nanoparticles (e.g., 15-20 nanometers) in the rat's brain following the opening and closing of the BBB.

To capture the iron oxide in the rat brain parenchyma, the BBB-opening treatment may be applied in a stable cavitation mode. Moreover, to avoid transition to an inertial cavitation mode, the BBB-opening treatment may be monitored using harmonic bubble activity emissions, ultra-harmonics, and broad emissions. The stable cavitation mode may be a more gentle BBB-opening treatment mode than the inertial cavitation mode.

Working in the stable cavitation mode may open the BBB for a time duration (e.g., minutes or hours) that allows the entry and accumulation of the iron oxide nanoparticles inside the brain parenchyma. The time duration may be short enough to be within BBB closure times, thus trapping the nanoparticles inside the brain parenchyma once the BBB is closed.

Together, FIGS. 4A-4C show how iron oxide might remain in a rat's brain twenty days following the treatment described above. Specifically, FIG. 4A depicts the rat brain immediately after the BBB-opening treatment. FIG. 4B depicts the rat brain following the administration of iron oxide (15-20 nanometers, and 26 milligram per kilogram). And FIG. 4C depicts the rat brain twenty days after the treatment. In each of the figures, the dotted rectangle indicates the treated hemisphere (18 sub-spots, 2 millimeter spacing). Additionally, the dark pixels in the dotted rectangles are the iron oxide particles that are trapped in the brain tissue.

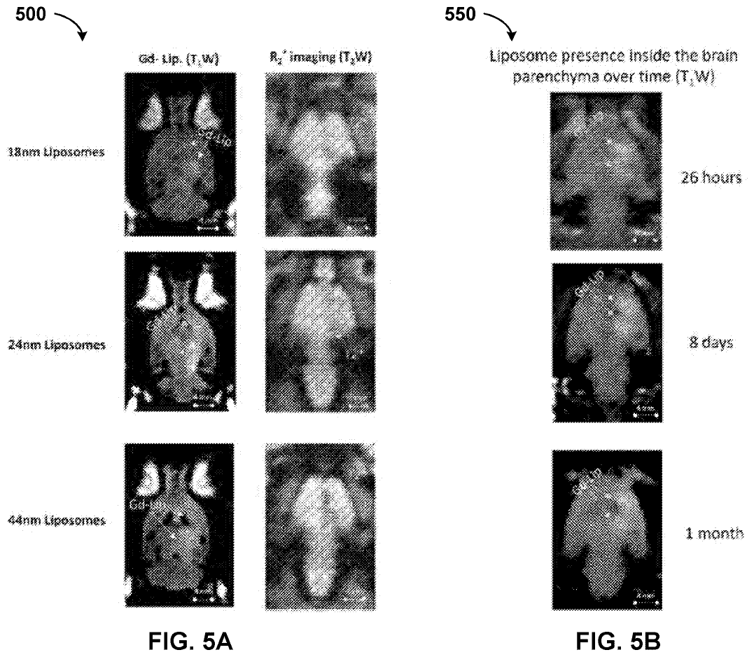

FIGS. 5A-5B are exemplary sets 500 and 550 of images that depict rat brains with different-sized liposomes introduced to respective brain parenchyma, in accordance with various embodiments of the present disclosure. The image set 500 of FIG. 5A depicts the rat brains shortly after introduction of the liposomes, and the image set 550 of FIG. 5B depicts the rat brains at various times thereafter. Images, such as the images of image sets 500 and 550, can be captured by attaching gadolinium (Gd) ions to liposomes. This can cause the liposomes to be visible and quantifiable in an MRI environment for about a month.

FIGS. 5A-5B together show that the systems and methods disclosed herein can be used to create reservoirs of different types of molecules. Whereas FIGS. 4A-4C show the presence of iron oxide in a rat brain, FIGS. 5A-5B show the presence of liposomes in a rat brain. The figures also suggest that the BBB-transition time of same-sized molecules may differ based on the substance of said molecules (e.g., iron oxide, liposomes). For example, 15-20 nanometers of iron oxide can be detected inside a brain parenchyma 20 minutes after injection, but liposomes of similar size may not be detectable until hours (e.g., 5 hours) after injection. Accordingly, treatment parameters may require tailoring based on the type of the molecule being delivered, in addition to the size of the molecule.

Referring now to FIG. 5A, the left-most column, labeled Gd-Lip. (T₁W), shows the concentration of gadolinium ions, in the light areas. The right-most column, labeled R₂* imaging (T₂W), shows treatment-related qualitative R₂* changes, in the dark areas. In FIG. 5B, the image set 550 shows the presence of 44 nanometer liposomes in a rat brain over a period of a month, first at 26 hours, then at 8 days, and finally at 1 month.

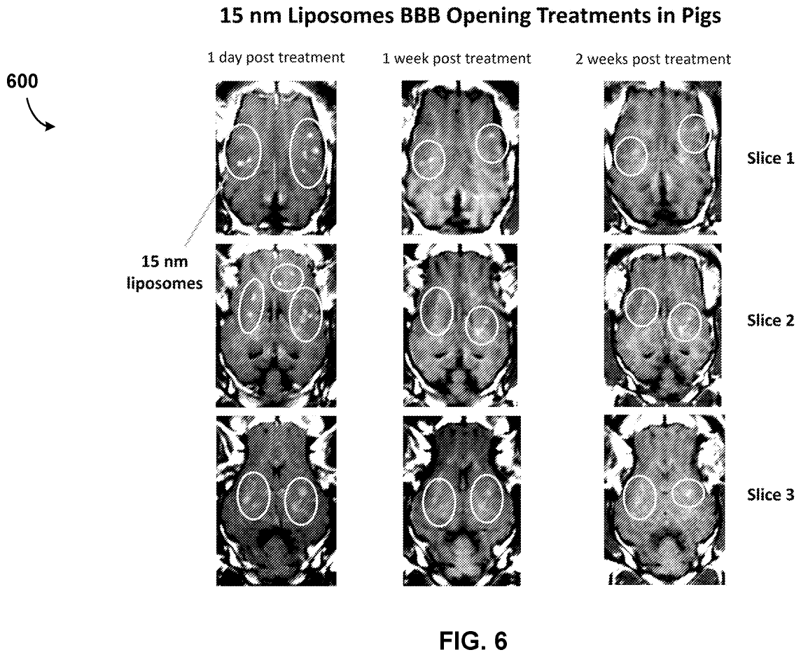

FIG. 6 is another exemplary set 600 of images that depict pig brains with liposomes introduced to respective brain parenchyma, in accordance with various embodiments of the present disclosure. The image set 600 shows three different image slices of pig brains with 15 nanometer liposomes introduced thereto, each image slice being taken at 1-day, 1-week, and 2-weeks post-treatment. As shown, the liposomes can be trapped inside the brain parenchyma of pigs for up to two weeks.

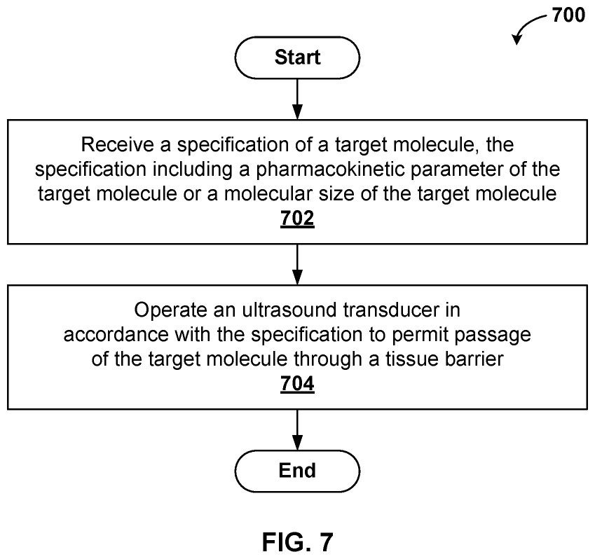

FIG. 7 is an exemplary block diagram 700 that depicts a process for controlling drug delivery through a tissue barrier, in accordance with various embodiments of the present disclosure. For illustrative purposes, the following discussion of the process refers to a single processor as executing the process. However, it may be executed by multiple processors, such as a processor of an ultrasound system 100 (see FIG. 1), of an imaging device (e.g., MRI apparatus 200 of FIG. 2), or of another electronic device.

The processor first receives (702) a specification of a target molecule. The specification includes a pharmacokinetic parameter of the target molecule, as well as a molecular size of the target molecule. The specification can also include, for example, chemical properties of the target molecule (e.g., a molecule type). After receiving the specification, the processor operates (704) an ultrasound transducer in accordance with the specification to permit passage of the target molecule through the tissue barrier in a manner that traps a target fraction of the target molecule behind the tissue barrier for a predetermined time period (e.g., 10 minutes, 1 hour, 2 days, or 4 weeks).

In some embodiments, the target molecule is injected before operation of the ultrasound transducer has been initiated. However, in some embodiments, the target molecule is not injected until after operation of the ultrasound transducer has been initiated.

In some embodiments, the transducer is operated in accordance with a sonication protocol to temporarily permeabilize the tissue barrier to the molecule, whereby the tissue barrier becomes impermeable to the molecule prior to its clearance into the bloodstream from behind the tissue barrier. Similarly, in some embodiments, the transducer is operated in accordance with a sonication protocol to partially permeabilize the tissue barrier to the molecule, whereby the target fraction remains behind the tissue barrier for the predetermined time period.

In some embodiments, the transducer is operated in accordance with a stable cavitation treatment regime. The stable cavitation treatment regime, for example, may be monitored to avoid transitioning to an inertial cavitation regime, using harmonic bubble activity emissions, ultra-harmonics, or broad emissions.

In some embodiments, the transducer is operated in accordance with certain levels of inertial cavitation. The inertial cavitation levels can be monitored using harmonic bubble activity emissions, ultra-harmonics, or broad emissions (incl., e.g., a combination thereof).

In some embodiments, the pharmacokinetic parameter comprises a diameter greater than 1 nanometer. In some embodiments, the target molecule has been altered to restrict its return through the tissue barrier following initial passage therethrough. For example, altering the target molecule can include attaching the target molecule to a complexing agent or to drug molecules. As another example, altering the target molecule can include changing a surface chemistry of the target molecule such that the target molecule forms aggregates after delivery of the target molecule through the tissue barrier.

In some embodiments, the processor causes, after operating the ultrasound transducer, a medical device to administer a drug for decreasing the permeability of the tissue barrier. For example, the medical device can be an infusion pump (e.g., a peristaltic pump or a syringe pump) configured to pump the drug into an infusion set attached to a catheter inserted into a patient. Alternatively, or additionally, the processor may provide a notification to a clinician requesting that the clinician provide the drug to the patient.

In some embodiments, the processor receives a ΔR₂* parameter from an MRI device (e.g., MRI apparatus 200 of FIG. 2) configured to detect the ΔR₂* parameter. The processor also determines the aforementioned predetermined time period based on the ΔR₂* parameter and the target fraction of the target molecule. The processor also operates the ultrasound transducer in accordance with the predetermined time period, as well as the aforenoted specification. In some implementations, the tissue barrier includes a BBB (e.g., of a patient). In some embodiments, the predetermined time period includes an estimated maximum amount of time for which the permeability of the tissue barrier allows the target molecule to pass through the tissue barrier (e.g., BBB).

Typically, the ΔR₂* parameter is used as a safety parameter during BBB-opening treatments to prevent vascular damage in the treatment area. However, ΔR₂* can also be utilized to predict the effectiveness of the BBB-opening treatment by serving as an index of the delivery of molecules with sizes similar to those of drugs used to treat various neurologic problems. This is discussed in more detail in M. Plaskin et al., Magnetic Resonance Imaging Analysis Predicts Nanoparticle Concentration Delivered to the Brain Parenchyma, Communications Biology 5, 964 (2022), which is incorporated by reference in its entirety.

In some embodiments, the target molecule includes a liposome, a viral vector, a small molecule drug (e.g., donepezil, galantamine, rivastigmine, memantine, suvorexant, carbidopa-levodopa, selegiline, rasagiline, safinamide, entacapone, benztropine, tolcapone, opicapone, nuplazid, istradefylline, or amantadine), a biologic drug, a gene therapy agent, a vaccine, an antisense oligonucleotide (ASO), a protein therapeutic, a modified mRNA agent, an RNAi agent, an antibody, an antibody-like molecule, or an antigen-binding fragment (e.g., of an antibody or an antibody-like molecule), a nonspecific clearing antibody (e.g., intravenous immunoglobulin aka IVIg), an anti-amyloid-β antibody (e.g., aducanumab, gantenerumab, lecanemab, or donanemab), an anti-tau antibody (e.g., semorinemab, gosuranemab, tilavonemab, or zagotenemab), or an anti-TREM2 antibody (e.g., AL002), an anti-alpha-synuclein antibody (e.g., Cinpanemab, Prasinezumab, Lu AF82422, ABBV-0805, or MEDI1341).

In some embodiments, wherein the target molecule comprises a molecule for treating a tumor (e.g., glioblastoma), a neurodegenerative disease (e.g., Alzheimer's disease, Parkinson's disease, Huntington's disease, amyotrophic lateral sclerosis, dementia with Lewy bodies, spinocerebellar ataxia, amyotrophic lateral sclerosis, frontotemporal diseases, multiple system atrophy, four-repeat tauopathy, or a prion disease), a central nervous system infection, or a congenital enzyme defect.

In general, functionality for facilitating an ultrasound procedure as described herein may be structured in one or more modules implemented in hardware, software, or a combination of both, whether integrated within a controller of the ultrasound system 100, an imager 112, or an administration system 126 (see FIG. 1), or provided by a separate external controller or other computational entity or entities. Such functionality may include, for example, one or more of the operations described herein (see, e.g., block diagram 500).

In addition, an ultrasound controller 108 or a controller associated with the administration system 126 (see FIG. 1) may include one or more modules implemented in hardware, software, or a combination of both. For embodiments in which the functions are provided as one or more software programs, the programs may be written in any of a number of high level languages such as PYTHON, FORTRAN, PASCAL, JAVA, C, C++, C#, BASIC, various scripting languages, and/or HTML. Additionally, the software can be implemented in an assembly language directed to the microprocessor resident on a target computer; for example, the software may be implemented in Intel 80x86 assembly language if it is configured to run on an IBM PC or PC clone. The software may be embodied on an article of manufacture including, but not limited to, a floppy disk, a jump drive, a hard disk, an optical disk, a magnetic tape, a PROM, an EPROM, EEPROM, field-programmable gate array (FPGA), or CD-ROM. Embodiments using hardware circuitry may be implemented using, for example, one or more FPGA, CPLD or ASIC processors.

The terms and expressions employed herein are used as terms and expressions of description and not of limitation, and there is no intention, in the use of such terms and expressions, of excluding any equivalents of the features shown and described or portions thereof. In addition, having described certain embodiments of this disclosure, it will be apparent to those of ordinary skill in the art that other embodiments incorporating the concepts disclosed herein may be used without departing from the spirit and scope of this disclosure. Accordingly, the described embodiments are to be considered in all respects as only illustrative and not restrictive.

In addition, the terms “approximately,” “roughly,” and “substantially” mean±10%, and in some embodiments, ±5%. Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, steps, or characteristics may be combined in any suitable manner in one or more examples of the technology. The headings provided herein are for convenience only and are not intended to limit or interpret the scope or meaning of the claimed technology.

1. A system for controlling drug delivery through a tissue barrier, the system comprising:
an ultrasound transducer for sonicating a target volume to cause disruption of the tissue barrier and thereby increase a permeability thereof; and
a controller configured to:
receive a specification of a target molecule, the specification including a pharmacokinetic parameter of the target molecule or a molecular size of the target molecule; and
operate the ultrasound transducer in accordance with the specification to permit passage of the target molecule through the tissue barrier in a manner that traps a target fraction of the target molecule behind the tissue barrier for a predetermined time period.

2. The system of claim 1, wherein the target molecule is injected before operation of the ultrasound transducer has been initiated.

3. The system of claim 1, wherein the target molecule is injected after operation of the ultrasound transducer has been initiated.

4. The system of claim 1, wherein the controller is configured to operate the transducer in accordance with a sonication protocol to temporarily permeabilize the tissue barrier to the molecule, whereby the tissue barrier becomes impermeable to the molecule prior to its clearance into the bloodstream from behind the tissue barrier.

5. The system of claim 1, wherein the controller is configured to operate the transducer in accordance with a sonication protocol to partially permeabilize the tissue barrier to the molecule, whereby the target fraction remains behind the tissue barrier for the predetermined time period.

6. The system of claim 1, wherein the controller is configured to operate the transducer in accordance with a stable cavitation treatment regime.

7. The system of claim 6, wherein the controller is configured to monitor the stable cavitation treatment regime to avoid transitioning to an inertial cavitation regime, using harmonic bubble activity emissions, ultra-harmonics, and/or broad emissions.

8. The system of claim 6, wherein the controller is configured to monitor the stable cavitation treatment regime to avoid transitioning to an inertial cavitation regime, using a combination of at least two of: harmonic bubble activity emissions, ultra-harmonics, and broad emissions.

9. The system of claim 1, wherein the specification specifies that the molecular size of the target molecule includes a diameter greater than 1 nanometer.

10. The system of claim 1, wherein the target molecule has been altered to restrict its return through the tissue barrier following initial passage therethrough.

11. The system of claim 10, wherein altering the target molecule comprises attaching the target molecule to a complexing agent or to drug molecules.

12. The system of claim 10, wherein altering the target molecule comprises changing a surface chemistry of the target molecule such that the target molecule forms aggregates after delivery of the target molecule through the tissue barrier.

13. The system of claim 1, further comprising a medical device configured to administer a drug for decreasing the permeability of the tissue barrier, wherein the controller is further configured to cause the medical device to administer the drug after operating the ultrasound transducer.

14. The system of claim 1, further comprising a magnetic resonance imaging (MRI) device configured to detect a ΔR₂* parameter:
the tissue barrier comprises a blood brain barrier (BBB); and
the controller is further configured to (i) receive the ΔR₂* parameter from the MRI device (ii) determine the predetermined time period based on the ΔR₂* parameter and the target fraction, and (iii) operate the ultrasound transducer further in accordance with the predetermined time period.

15. The system of claim 14, wherein the predetermined time period comprises an estimated maximum amount of time for which the permeability of the tissue barrier allows the target molecule to pass through the tissue barrier.

16. The system of claim 1, further comprising a device configured to administer an acoustic agent in the form of microbubbles, nanobubbles, or phase-shift droplets.

17. The system of claim 1, wherein the target molecule comprises a liposome, a viral vector, a small molecule drug, a biologic drug, a gene therapy agent, a vaccine, an antisense oligonucleotide (ASO), a protein therapeutic, a modified mRNA agent, an RNAi agent, an antibody, an antibody-like molecule, or an antigen-binding fragment, a nonspecific clearing antibody, an anti-amyloid-β antibody, an anti-tau antibody, an anti-TREM2 antibody, or an anti-alpha-synuclein antibody.

18. The system of claim 1, wherein the target molecule comprises a molecule for treating a tumor, a neurodegenerative disease, a central nervous system infection, or a congenital enzyme defect.

19. The system of claim 1, wherein the controller is configured to operate the transducer in certain levels of inertial cavitation treatment regime.

20. The system of claim 19, wherein the controller is configured to monitor the inertial cavitation levels using harmonic bubble activity emissions, ultra-harmonics, or broad emissions.

21. The system of claim 19, wherein the controller is configured to monitor the inertial cavitation levels using a combination of at least two of harmonic bubble activity emissions, ultra-harmonics, and broad emissions.

22. The system of claim 1, wherein the specification further includes a chemical property of the target molecule.
